(19) United States
(12) Patent Application Publication
(10) Pub. No.: US 2023/0249200 A1
(43) Pub. Date: Aug. 10, 2023

(54) SPRAY HEAD AND DEVICE FOR DISPENSING A FLUID PRODUCT COMPRISING SUCH A HEAD

(71) Applicant: APTAR FRANCE SAS, LE NEUBOURG (FR)

(72) Inventor: Matthieu SAVALLE, OISSEL (FR)

(73) Assignee: APTAR FRANCE SAS, LE NEUBOURG (FR)

(21) Appl. No.: 18/004,499

(22) PCT Filed: Jul. 5, 2021
(86) PCT No.: PCT/FR2021/051226

§ 371 Date: Jan. 6, 2023

(30) Foreign Application Priority Data
Jul. 7, 2020 (FR) FR2007187

(51) Int. Cl. B05B 1/34; B05B 11/02; A61M 15/08; B33Y 80/00

(52) U.S. Cl. CPC B05B 1/3478; B05B 1/3426; B05B 11/02; A61M 15/08; B33Y 80/00

(57) ABSTRACT

The invention relates to a spray head for a device for dispensing a fluid product, having a body provided with a spray orifice, a central channel being connected to the spray orifice via a spray profile having a vortex chamber arranged directly upstream of the spray orifice, the spray profile having at least two curved channels extending from the central channel to the vortex chamber, each curved channel connected non-radially to the vortex chamber, to form a spray exiting the spray orifice, each curved channel having a convex shape, diverging laterally outwards from the central channel, and then from an inflection point, returning laterally inwards towards the vortex chamber.

The present invention relates to a spray head, its method of manufacture, and to a device for dispensing a fluid product, comprising such a head.

More particularly, the present invention relates to a spray head which is made as a single piece.

Generally, to produce a spray profile upstream from the spray orifice, an insert is fitted into the spray head.

That type of device may present certain drawbacks. This means that, according to the method of manufacture, at least two parts need to be manufactured, and also an assembly step is then necessary. In addition, due to the manufacturing tolerances of the parts to be assembled (the spray head and the insert), the spray profile is not always strictly identical, and the performance of the spray is not always reproducible.

Documents WO2008015409 and WO8905195 describe devices of the state of the art.

The present invention aims to provide a spray head for a device for dispensing a fluid product, which does not have the above-mentioned drawbacks.

More particularly, the present invention aims to provide a spray head with a spray profile that is made as a single piece.

The present invention also aims to provide such a spray head that can be produced in a single manufacturing step.

The present invention also aims to provide such a spray head that is simple and inexpensive to manufacture and to assemble.

The present invention thus provides a spray head for a device for dispensing a fluid product, comprising a body provided with a spray orifice, a central channel being connected to said spray orifice via a spray profile comprising a vortex chamber arranged directly upstream of said spray orifice, said spray profile comprising at least two curved channels extending from said central channel to said vortex chamber, each curved channel connected non-radially to said vortex chamber, to form a spray exiting said spray orifice, each curved channel having a convex shape, diverging laterally outwards and axially upwards from said central channel, and then from an inflection point, returning laterally inwards towards said vortex chamber.

Advantageously, said spray head is of the nasal type, said central channel extending axially and said spray orifice being orientated axially.

Advantageously, said at least two curved channels open out into said vortex chamber while being regularly distributed over the periphery of said vortex chamber.

Advantageously, each curved channel is rounded in shape, without any sharp corners.

Advantageously, the cross-section of each curved channel is reduced progressively between said central channel and said vortex chamber.

Advantageously, said at least two curved channels are substantially identical in shape, dimension and curvature, between said central channel and said vortex chamber.

In a variant, said at least two curved channels are different in shape, dimension and/or curvature, between said central channel and said vortex chamber.

Advantageously, said spray profile comprises two curved channels.

Advantageously, said spray profile comprises three curved channels.

The present invention also provides a device for dispensing a fluid product, comprising: an actuation portion; a reservoir containing a fluid product; a spray head provided with a spray orifice; and a dispenser means for dispensing at least a portion of said fluid product through said spray orifice during actuation, said spray head being made as described above.

Advantageously, said reservoir contains a single dose of fluid product dispensed in a single actuation of the device.

In a variant, said reservoir contains at least two doses of fluid product dispensed during a plurality of successive actuations of the device.

Advantageously, said actuation portion comprises two lateral presser elements mounted to pivot on said spray head, each lateral presser element comprising a respective cam portion each comprising a respective cam surface co-operating with said reservoir to move it axially upwards while the device is being actuated.

The present invention also provides a method of manufacturing a spray head as described above, said method producing said spray head by additive printing.

These features and advantages and others of the present invention will appear more clearly during the following detailed description, made in reference to the accompanying drawings, given as non-limiting examples, and wherein:

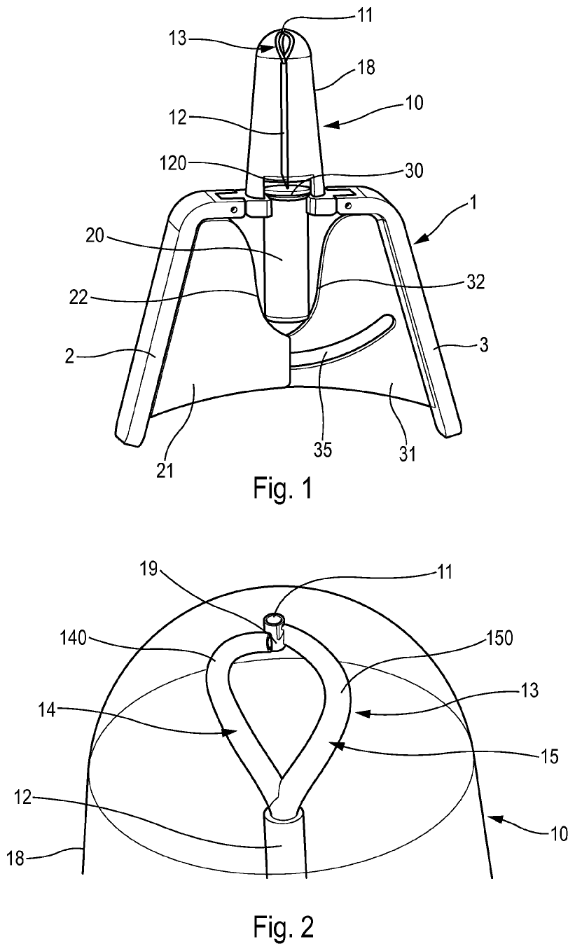

FIG. 1 is a diagrammatic side view of a nasal fluid product dispenser device in an advantageous embodiment;

FIG. 2 is a detailed diagrammatic perspective view of a spray head according to an advantageous first embodiment;

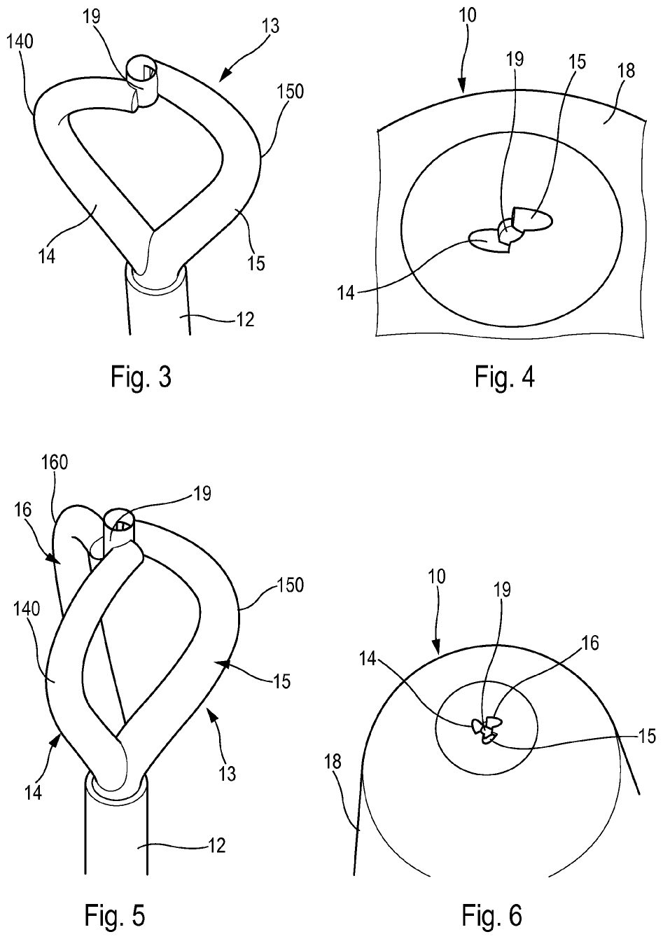

FIG. 3 is a view similar to the view in FIG. 2;

FIG. 4 is a diagrammatic plan view, showing a cross-section of the spray head in FIGS. 2 and 3;

FIG. 5 is a view similar to the view in FIG. 3, showing a second advantageous embodiment;

FIG. 6 is a view, similar to the view in FIG. 4, showing a cross-section of the spray head in FIG. 5;

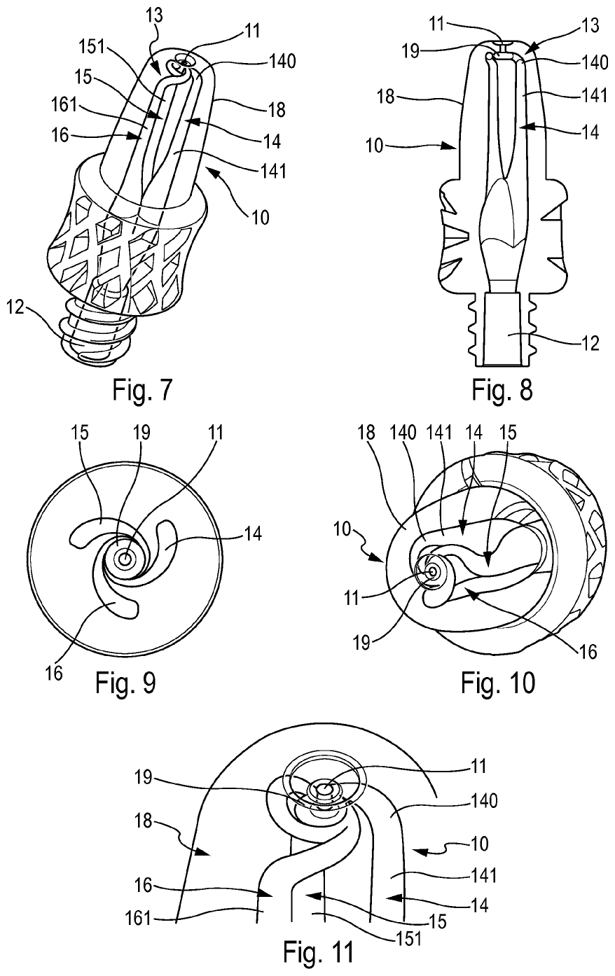

FIG. 7 is a transparent diagrammatic perspective view of a spray head according to an advantageous third embodiment;

FIG. 8 is a diagrammatic side view of the spray head in FIG. 7;

FIG. 9 is a diagrammatic plan view of the head in FIG. 7,

FIG. 10 is a transparent diagrammatic perspective view of the spray head in FIG. 7; and

FIG. 11 is a diagrammatic and fragmentary perspective view of the spray head in FIG. 7.

The terms “proximal” and “distal” are relative to the spray orifice. The terms “axial” and “radial” are relative to the longitudinal central axis of the device. The terms “top”, “bottom”, “upper” and “lower” refer to the upright position of the device represented in FIG. 1.

FIG. 1 shows an example of a nasal fluid product dispenser device to which the present invention applies. In this example, the device is a single-dose device in which all of the dose of fluid product is dispensed in a single actuation. It should be observed that the present invention could also be adapted to other types of devices, e.g., such as devices of the two-dose or multi-dose type, containing two or more doses of fluid product to be dispensed during two or more successive actuations of the device.

In the embodiment in FIG. 1, the device comprises an actuation portion 1, a spray head 10 comprising a body 18 provided with a spray orifice 11, a reservoir 20 containing the fluid product to be dispensed and dispenser means 30 for dispensing a dose of fluid product while the device is being actuated.

The spray head 10, in this embodiment, of the nasal type, comprises a central channel 12 connected to said spray orifice 11 via a spray profile 13. The spray orifice 11 serves to dispense a dose of fluid product out from said spray head 10 while the device is being actuated by a user. A cannula 120 is arranged at the distal end of the central channel 12.

The reservoir 20 is connected to the spray head 10 in any appropriate manner. In the example represented in FIG. 1, the reservoir 20 contains a single dose of fluid product dispensed in a single actuation of the device. Typically, as can be seen in the figures, the reservoir 20 may be formed by a hollow and blind body, comprising a single proximal opening.

In another embodiment that is not shown in the drawings, the reservoir need not be formed by a hollow and blind body that includes only one opening, but may be formed by a hollow cylinder that is open axially at both ends. The cylinder would thus be closed at the proximal end by a first stopper and at the distal end by a second stopper, the volume defined between said two stoppers containing the fluid product to be dispensed.

In another variant, not shown, the reservoir 20 could be a syringe, on which the spray head is attached by any appropriate means, for example a standard rotary fastening of the Luer-lock type. In the embodiment in FIGS. 7 to 11, the spray head 10 comprises a thread adapted to be attached to a syringe fastening of the Luer-lock type.

In the embodiment shown in FIG. 1, which shows a single dose, the dispensing means 30 comprise a stopper/piston. This stopper/piston 30 closes the reservoir in sealed manner before actuation, and is adapted to be perforated by the cannula 120 at the start of actuation for connecting the reservoir 20 to the spray orifice 11. Continued actuation moves said stopper/piston in the reservoir 20, thus causing the fluid product to be expelled through the cannula 120 to the spray orifice 11. In a multi-dose device, the dispenser means could comprise a pump or a metering valve.

In this embodiment, the actuation portion 1 comprises two lateral presser elements 2, 3, which also support the user's fingers during actuation. These two lateral presser elements 2, 3 are mounted to pivot on the spray head 10. Each lateral presser element 2, 3 comprises a respective cam portion 21, 31, said cam portions 21, 31 being connected to each other to allow the lateral presser elements to pivot, for example via a lug (not shown) in the cam portion 21 sliding in a groove 35 in the cam portion 31. Each cam portion 21, 31 comprises a respective cam surface 22, 32 co-operating with the reservoir 20, in particular with its radially outer distal edge, as can be seen in FIG. 1. During actuation, the user compresses the lateral presser elements 2, 3 towards each other, which causes them to pivot, the cam surfaces 22, 32 then push the reservoir 20 axially upwards to dispense a dose of fluid product.

The embodiment shown therefore shows a lateral actuation of the device, but it is understood that the present invention could also apply to a conventional actuation of the axial type.

According to the invention, the spray head 10 is made as a single piece by additive printing.

This makes it possible to produce a spray profile 13 which has a complex shape, and is difficult or even impossible to produce by moulding and/or assembling.

According to the invention, the spray profile 13 comprises at least two curved channels 14, 15, 16 extending from the central channel 12 to a vortex chamber 19 arranged directly upstream from the spray orifice 11. FIGS. 2 to 4 show a first embodiment with two curved channels 14, 15, FIGS. 5 and 6 show a second embodiment with three curved channels 14, 15, 16, and FIGS. 7 to 11 show a third embodiment with three curved channels 14, 15, 16. It is possible to envisage a spray profile 13 with more than three curved channels, for example four.

In the embodiment shown, which shows a nasal spray head, the central channel 12 is axial, as is the spray orifice 11.

It should be observed that the present invention could also apply to a device comprising an oral dispenser head, with a spray orifice which is radially orientated.

Each curved channel 14, 15, 16 is connected non-radially to the vortex chamber 19, thus generating a vortex of the flows leaving said curved channels in said vortex chamber 19, thus forming a spray at the outlet of the spray orifice 11.

Preferably, the at least two curved channels 14, 15, 16 open out into the vortex chamber 19, while being regularly distributed over the periphery thereof. Thus, in the embodiment in FIGS. 2 to 4, the two curved channels 14, 15 open out diametrically opposite each other into the vortex chamber 19, and in the embodiments in FIGS. 5 and 6 and in FIGS. 7 to 11, the three curved channels 14, 15, 16 open out into the vortex chamber 19, while being spaced apart from each other by 120°. In a variant, however, it is possible to envisage opening out the curved channels into the vortex chamber while being distributed in a non-uniform manner, in particular if the dimensions of the various curved channels are different.

As can be seen, in particular, in FIGS. 2, 3, 5, 7, 8, 10 and 11, each curved channel 14, 15, 16 is convex in shape, diverging laterally outwards and axially upwards from the central channel 12, then from an inflection point 140, 150, 160, returning laterally inwards towards the vortex chamber 19. Said inflection points 140, 150, 160 advantageously form the radially outermost portions of said curved channels 14, 15, 16.

In the embodiment in FIGS. 7 to 11, each of the inflection points 140, 150, 160 is formed in an upper axial portion of an axial portion of the channel 141, 151, 161 that extends substantially axially over a large portion of the height of the spray head 10. Thus, as can be seen, in particular, in FIGS. 7, 8 and 10, the three curved channels 14, 15, 16 move laterally away from the central channel 12, then extend parallel to each other over the axial portion of the channel 141, 151, 161 as far as the vicinity of the vortex chamber 19, to finally return laterally in a non-radial manner towards the inside.

Advantageously, each curved channel 14, 15, 16 is rounded in shape, without any sharp corners. This makes it possible in particular to limit the pressure drops, such that the flows coming from the curved channels penetrate into the vortex chamber 19 with maximum energy, thus generating a spray of excellent quality at the outlet of the spray orifice 11.

Advantageously, the cross-section of each curved channel 14, 15, 16 is reduced progressively between the central channel 12 and the vortex chamber 19. This makes it possible in particular to accelerate the flows flowing in said curved channels, optimising their speed when they open out into the vortex chamber 19.

Advantageously, said at least two curved channels 14, 15, 16 are substantially identical in shape, dimension and curvature, between the central channel 12 and the vortex chamber 19. Nevertheless, in a variant, it is possible to envisage making curved channels of different shapes, dimensions, in particular length and section, and of different curvatures, thus generating different flow rates in each curved channel. Associated with a non-uniform distribution of the curved channels around the vortex chamber, this could make it possible to produce sprays of any required shape.

As described above, the invention could also apply to a device of the dual-dose type. In that configuration, the contents of the reservoir would be dispensed during two successive actuations. Document WO 2014/147329 describes an example of a two-dose device. More generally, the present invention applies to all types of single-dose, two-dose, or multi-dose devices.

According to an advantageous aspect of the invention, the spray head 10 is manufactured by additive printing or 3D printing. This method of manufacture makes it possible to produce complex single-piece shapes, such as the spray head 10 including the spray profile 13 with the curved channels 14, 15, 16, as a single piece, without assembling one or more inserts, and with perfectly reproducible performances from one spray head to the other.

The present invention provides several advantages, and in particular:

it improves the reproducibility of the spray, each spray head having an identical spray profile;

it avoids having to assemble two or more parts which are manufactured separately, eliminating a manufacturing step and an assembly step;

it makes it possible to produce any complex shapes required, by using additive printing;

it makes it possible to optimise the spray properties thanks to an optimised geometry of the spray profile.

Naturally, other variant embodiments may also be envisaged, without going beyond the scope of the present invention, as defined by the accompanying claims.

1. spray head for a device for dispensing a fluid product, comprising a body provided with a spray orifice, a central channel being connected to said spray orifice via a spray profile comprising a vortex chamber arranged directly upstream of said spray orifice, characterised in that said spray profile comprises at least two curved channels extending from said central channel to said vortex chamber, each curved channel connected non-radially to said vortex chamber, to form a spray exiting said spray orifice, each curved channel having a convex shape, diverging laterally outwards from said central channel, and then from an inflection point, returning laterally inwards towards said vortex chamber.

2. The spray head according to according claim 1, wherein said spray head is of the nasal type, said central channel extending axially and said spray orifice being orientated axially.

3. The spray head according to claim 1, wherein said at least two curved channels open out into said vortex chamber while being regularly distributed over the periphery of said vortex chamber.

4. The spray head according to claim 1, wherein each curved channel is rounded in shape without any sharp corners.

5. The spray head according to claim 1, wherein the cross-section of each curved channel is reduced progressively between said central channel and said vortex chamber.

6. The spray head according to claim 1, wherein said at least two curved channels are substantially identical in shape, dimension and curvature between said central channel and said vortex chamber.

7. The spray head according to claim 1, wherein the said at least two curved channels are different in shape, dimension and/or curvature between said central channel and said vortex chambe.

8. The spray head according to claim 1, wherein said spray profile comprises two curved channels.

9. The spray head according to claim 1, wherein said spray profile comprises three curved channels.

10. The device for dispensing a fluid product, comprising an actuation portion; a reservoir containing a fluid product; a spray head provided with a spray orifice; and a dispenser means for dispensing at least a portion of said fluid product through said spray orifice during actuation, characterised in that said spray head is manufactured according to claim 1.

11. The device according to claim 10, wherein said reservoir contains a single dose of fluid product dispensed in a single actuation of the device.

12. The device according to claim 10, wherein said reservoir contains at least two doses of fluid product dispensed during a plurality of successive actuations of the device.

13. The device according to claim 10, wherein said actuation portion comprises two lateral presser elements mounted to pivot on said spray head, each lateral presser element comprising a respective cam portion, each comprising a respective cam surface co-operating with said reservoir to move it axially upwards while the device is being actuated.

14. The method of manufacturing a spray head according to claim 1, characterised in that said method comprises making said spray head by additive printing.
